(19) United States
(12) Patent Application Publication
(10) Pub. No.: US 2009/0203118 A1
(43) Pub. Date: Aug. 13, 2009

(54) OPTICAL SYSTEM INCLUDING NANOSTRUCTURES FOR BIOLOGICAL OR CHEMICAL SENSING

(75) Inventors: Fu-Jen Kao, Kaohsiung (TW); Hiroyuki Takei, Saitama (JP); Randolph Storer, Hillsborough, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: LamdaGen Corporation, Burlingame, CA (US)

(21) Appl. No.: 12/421,867

(22) Filed: Apr. 10, 2009

Related U.S. Application Data
(63) Continuation of application No. 11/473,458, filed on Jun. 22, 2006.
Application No. 11/473,458 is a division of application No. 10/901,916, filed on Jul. 28, 2004, now abandoned.
(60) Provisional application No. 60/490,781, filed on Jul. 29, 2003.

(51) Int. Cl. C12M 1/34

(52) U.S. Cl. 435/288.7

(57) ABSTRACT

An analytical device is disclosed. In one embodiment, the analytical device includes a three-dimensional substrate structure comprising a three-dimensional surface. A plurality of noble metal nanoparticles are on the three-dimensional substrate structure. A plurality of capture agents are on the noble metal nanoparticles.

### CROSS-REFERENCES TO RELATED APPLICATIONS

This application is a non-provisional of U.S. Provisional Patent Application No. 60/490,781, filed on Jul. 29, 2003, which is herein incorporated by reference in its entirety for all purposes.

### BACKGROUND OF THE INVENTION

Nanoparticles comprising noble metals exhibit sharp absorption in the UV-visible region due to the resonant oscillation of free electrons or a localized surface plasmon. Excitation of the localized surface plasmon results in the generation of intense electromagnetic fields in the immediate vicinity of the nanoparticles. The resonance frequency of the localized surface plasmon depends on the refractive index within the local electromagnetic fields. A number of workers have used this phenomenon as a sensing mechanism, for example, in a biosensor whereby nanoparticles coated with capturing biomolecules undergo a color change after other biomolecules are bound to the capturing molecules.

A color change is particularly strong when binding events between two types of biomolecules results in aggregation of nanoparticles, particularly gold nanoparticles. For example, each gold nanoparticle in a set of gold nanoparticles can be coated with a DNA fragment of one sequence and another set of gold nanoparticles can be coated with another DNA fragment with the complimentary sequence. Alternatively, an antibody such as immunoglobulin G can bridge two gold nanoparticles coated with the corresponding antigen.

These methods, however, have the following disadvantages: (1) they cannot provide quantitative kinetic data concerning the binding event, (2) they can detect only a biomolecule that can form more than one bond simultaneously, (3) they cannot be reused, (4) it is difficult to multiplex the system to monitor more than one type of target biomolecule simultaneously, and (5) they cannot monitor a series of biomolecular binding events. The first disadvantage is a particularly limiting factor when detailed knowledge of interactions is now highly desired. In proteomics, it is desirable to study and understand detailed networks of protein interactions. With development of new drugs, one must know precisely how fast a target molecule binds to a proposed capture agent and how fast it unbinds to the capture agent.

While gold nanoparticles have been used to detect target molecule binding events, conventional analysis methods and apparatuses all suffer from low sensitivity. A means to increase the surface area by using a three-dimensional substrate structure would be desirable. The structure is desirable uniform and is desirably prepared with a high degree of reproducibility. Other changes to improve sensitivity would also be desirable.

### SUMMARY OF THE INVENTION

Embodiments of the invention are directed to analytical devices, analytical apparatuses, and methods.

One embodiment of the invention is directed to an analytical device comprising: (a) a three-dimensional substrate structure comprising a three-dimensional surface; (b) a plurality of noble metal nanoparticles on the three-dimensional substrate structure; and (c) a plurality of capture agents on the noble metal nanoparticles.

One embodiment of the invention is directed to an analytical apparatus comprising: (a) an agitating device; (b) an analytical device comprising a detection region, wherein the detection region comprises a plurality of noble metal nanoparticles and capture agents coupled to the noble metal nanoparticles; (c) an optical emitter capable of providing a first signal to the detection region; and (d) an optical detector capable of detecting a second signal from the detection region, wherein the agitating device is capable of agitating (and mixing) a fluid comprising target molecules in the detection region.

Another embodiment of the invention is directed to a method comprising: (a) providing a three-dimensional substrate structure comprising a three-dimensional surface; (b) depositing a plurality of noble metal nanoparticles on the three-dimensional surface of the three-dimensional substrate structure; (c) attaching a plurality of capture agents to the noble metal nanoparticles; (d) contacting a fluid comprising a target analyte to the noble metal nanoparticles while the noble metal nanoparticles are on the three-dimensional surface; (e) directing a first optical signal to the noble metal nanoparticles; and (f) receiving a second optical signal from the noble metal nanoparticles after (e).

Another embodiment of the invention is directed to a method comprising: (a) providing a substrate structure comprising a surface; (b) depositing a plurality of noble metal nanoparticles on the surface of the substrate structure; (c) attaching a plurality of capture agents to the noble metal nanoparticles; (d) contacting a fluid comprising a target analyte to the noble metal nanoparticles while the noble metal nanoparticles are on the three-dimensional surface; (e) directing a first optical signal to the noble metal nanoparticles; (f) receiving a second optical signal from the noble metal nanoparticles after (e); and (g) mixing the fluid while (e) and (f) are being performed.

These and other embodiments of the invention are described in further detail below with reference to the Figures and the Detailed Description.

### BRIEF DESCRIPTION OF THE DRAWINGS

FIG. 1 shows a schematic diagram of an analytical apparatus according to an embodiment of the invention.

FIG. 2 shows a schematic diagram of a portion of an analytical device according to an embodiment of the invention.

FIG. 3 shows a schematic drawing showing how incident light responds when directed to an analytical device.

FIG. 4(a) shows a schematic diagram showing a plurality of nanoparticles on a three-dimensional substrate, each nanoparticle having a capture agent bound to it.

FIG. 4(b) shows a schematic diagram showing a plurality of nanoparticles on a three dimensional substrate, each nanoparticle having a capture agent bound to it. Target molecules are captured by the capture agents that are attached to the nanoparticles.

FIG. 4(c) shows a schematic diagram of spectrum shift changes that occur when target molecules bind to capture agents attached to the nanoparticles.

FIG. 5(a)-5(c) show how the three-dimensional substrate structure may comprise a collection of particles that are larger than the noble metal nanoparticles.

FIG. 6(a) shows a schematic representation of an analytical device including a three-dimensional substrate structure in the form of a fabric (or alternatively a membrane), wherein nanoparticles are coupled to the surfaces of the fibers in the fabric. The fabric may comprise nanotubes, nanowires, and nanomeshes.

FIG. 6(b) shows a schematic representation of an analytical device including a substrate including many nanochannels and nanoparticles on the substrate.

FIG. 6(c) shows a schematic representation of an analytical device including a substrate including funnels and cylinders, with nanoparticles in the funnels (e.g., nanofunnels) and cylinders (e.g., nanocylinders).

FIG. 7(a) shows a schematic representation of an analytical apparatus including an agitating device.

FIG. 7(b) shows an end view of a fiber bundle used in the analytical apparatus.

FIG. 7(c) shows a side cross-sectional view of an agitating device as it is used to agitate a fluid.

FIG. 8(a) shows an analytical apparatus according to another embodiment of the invention.

FIG. 8(a)-8(d) show how a fiber bundle can move to agitate a fluid.

FIG. 8(e) shows an end view of a fiber bundle according to an embodiment of the invention.

FIG. 9(a)-9(b) show another analytical apparatus according to another embodiment of the invention.

FIG. 9(c) shows an end view of another fiber bundle according to another embodiment of the invention.

FIG. 10(a) shows another analytical apparatus according to another embodiment of the invention.

FIG. 10(b) shows an end view of another fiber bundle according to an embodiment of the invention.

FIG. 10(c) shows a side view of an agitating device according to another embodiment of the invention.

FIG. 11(a) shows another analytical apparatus according to another embodiment of the invention.

FIG. 11(b) shows a side view of a fluid as it is being agitated.

FIG. 11(c) shows an end view of an optical fiber bundle and an agitating device.

FIG. 12(a) shows an optical fiber bundle that can be connected to multiple light emitting diodes.

FIG. 12(b) shows an end view of the fiber bundle.

FIG. 12(c) shows an optical spectrum.

FIGS. 13 and 14 show plots of wavelength vs. time.

FIG. 15 shows a plot of wavelength vs. time for an embodiment of the invention.

FIG. 16 shows a photograph of an analytical device according to an embodiment of the invention. As shown, the substrate of the analytical device has a bumpy, three-dimensional surface. Nanoparticles (not readily visible) are on the three-dimensional surface.

FIG. 17 shows a photograph of an analytical device including a substrate including a flat surface.

### DETAILED DESCRIPTION

Embodiments of the invention include methods for preparing multiple layers of noble metal nanoparticles that exhibit a pronounced absorption spectrum when exposed to light. Embodiments of the invention are also directed to analytical apparatuses and analytical devices that exploit the optical properties of the noble metal nanoparticles for the detection of binding events between molecules. Using embodiments of the invention, biochemical assays can be performed quickly, accurately, and efficiently.

Some embodiments of the invention are directed to various ways to enhance the interactions between target molecules in a fluid and capture agents in an analytical device. In some embodiments, optical fibers in an analytical apparatus direct light to and receive light from multiple layers of noble metal nanoparticles and/or nanoparticles on a three-dimensional surface of a three-dimensional substrate structure. A liquid sample containing target biomolecules can be actively mixed in a mixing region of an analytical device. This helps the target biomolecules interact with the capture agents attached to the noble metal nanoparticles so that binding (if any) can occur between the target biomolecules and the capture agents.

While gold is the most desirable material due to its stability, other noble metals such as silver, platinum, palladium, etc. may be used in the noble metal nanoparticles as well. Further, any alloys of composites including these noble metals may be included in the noble metal nanoparticles. Also, more than one type of nanoparticles could be adsorbed on the three-dimensional surface. For example, gold and silver nanoparticles can be adsorbed on the same three-dimensional surface of the substrate.

The noble metal nanoparticles may be small. For example, the noble metal nanoparticles may have a diameter less than about 350 (e.g., 275) nanometers, preferably less than about 140 nanometers.

The three-dimensional substrate structures having a three-dimensional surface may comprise any suitable material including silicon, polystyrene, glass, etc. The substrate may even include a noble metal such as gold.

The three-dimensional substrate structure may be in the form of a plurality of particles, a fabric (woven or nonwoven), a porous body, a substrate including an undulating surface, a substrate including nanochannels, a substrate including hollow regions such as cones or cylinders, etc. In some embodiments, the three-dimensional substrate may include a membrane, elliptical spheroids, etc. It may include nanopores, nanowires, nanochannels, and nanowire meshes.

The three-dimensional substrate structures may be formed by any suitable process. For example, in one embodiment, heating a planar substrate until the upper surface of the substrate wrinkles and warps may form a substrate including an undulating surface. This produces a substrate including a three-dimensional undulating surface with peaks and valleys. In other embodiments, the three-dimensional substrate structure may be formed using a process such as an injection molding process or the like.

Two aspects of embodiments of the invention will be discussed. The first relates to the use of three-dimensional substrate structures including three-dimensional surfaces. The second relates to analytical apparatuses that use agitating devices. It is understood that embodiments of the invention can include either of these aspects without the other. However, preferred embodiments use both aspects together. It is understood that any of the features that are shown in the specific embodiments may be combined with any other features in any other embodiments without departing from the spirit and scope of the invention.

The capture agents and target molecules that are used in embodiments of the invention can include any biological or chemical entity. For example, the capture agents and target molecules can include nucleic acids (e.g., RNA, DNA), proteins, polypeptides, oligonucleotides, chemical compounds, drugs, drug candidates, etc.

I. Three-Dimensional Substrate Structures Including Three-Dimensional Surfaces

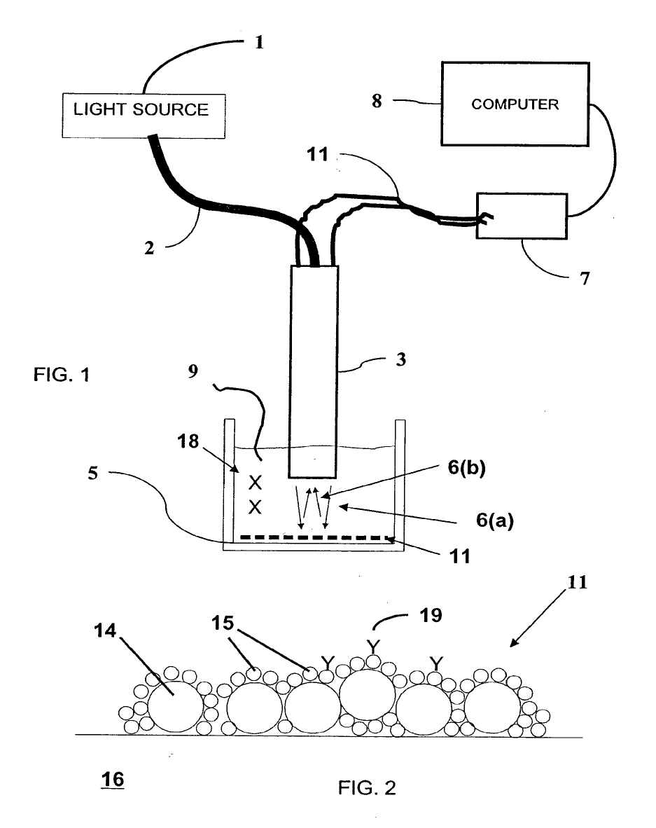

FIG. 1 shows a schematic illustration of an analytical apparatus according to an embodiment of the invention. The analytical apparatus includes an analytical device 11 inside of a container 5 containing a fluid 9. The fluid 9 in the container 5 may comprise target molecules 18. A fiber bundle 3 directs incident light 6(a) including a first optical signal to the analytical device 11 and receives reflected light 6(b) including a second optical signal from the analytical device 11.

The fiber bundle 3 includes portions of at least one emitter optical fiber 2 and at least one receiver optical fiber 11. The at least one emitter optical fiber 2 is coupled to an optical emitter 1, while the at least one optical receiver fiber is coupled to an optical detector 7. The fiber bundle 3 advantageously allows light to be directed to and received from a localized area on the analytical device 11. A computer 8 may be coupled to the optical detector 7. The computer 8 may be a general-purpose personal computer, laptop computer, server computer, or a specifically designed ASIC (application specific circuit) chip.

For clarity of illustration, many of the described examples show a single well or container containing a single fluid. However, it is understood that many such wells or containers can be used so that parallel assays like those that use a 96 well plate (or larger) can be performed. In such embodiments, there may be, for example, a single optical fiber bundle that sequentially detects binding events in different wells. Alternatively, there may be multiple optical fiber bundles that detect binding events in multiple wells in parallel.

FIG. 2 shows a schematic illustration of an analytical device 11 according to an embodiment of the invention. FIG. 2 shows a three-dimensional substrate structure in the form of a plurality of non-noble metal particles 14. The surfaces 14(a) of the non-noble metal particles 14 (e.g., polystyrene, silicon, glass, graphite, etc.) may be three-dimensional surfaces. A plurality of noble metal nanoparticles 15 (e.g., gold, silver, platinum, alloys thereof, etc.) are on the three-dimensional surfaces 14(a) of the non-noble metal nanoparticles 14. The non-noble metal nanoparticles 14 and the noble metal nanoparticles 15 may both be present on the bottom region of the container 5. Capture agents 19 may be present on the noble metal nanoparticles 15 and can interact with the target molecules 18 in the fluid 9.

An exemplary method according to an embodiment of the invention can be described with reference to FIGS. 1 and 2. The capture agents 19 attached to the noble metal nanoparticles 15 may comprise antibodies and the target molecules 18 may be potential candidate drug molecules 18 that bind to the antibodies. The optical emitter 1 transmits light of a particular wavelength through the optical emitter fiber 2 to the fiber bundle 3, and then through a tip (not shown) of the optical emitter fiber 2. The surface-adsorbed noble metal particles 14 are at the bottom of the container 5 and are irradiated with the light (including a first optical signal) from the fiber bundle 3. Reflected light (including a second optical signal) of a different characteristic than the light that was used to irradiate the noble metal nanoparticles 14 is then received by the fiber bundle 3. The reflected light (including the second optical signal) is sent to the optical detector 7 (e.g., a spectrometer). The computer 8 then processes and displays data received from the optical detector 7 in a user friendly and/or user defined format. In the absence of any target molecules 18, the maximum absorption wavelength may be X.

If the capture agents capture the target molecules 18, then the wavelength of the maximum absorption peak associated with the noble metal nanoparticles 15 shifts to a wavelength Y. If no binding occurs between the target molecules 18 and the capture agents 19, then there is no shift in the maximum absorption peak-wavelength X. In this way, binding events, if any, can be detected between target molecules 18 and capture agents 19.

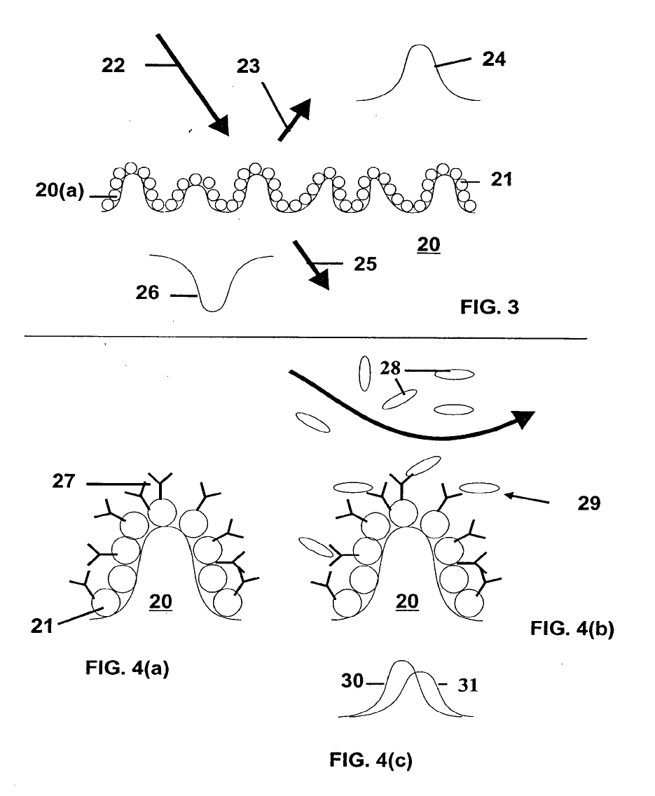

FIG. 3 shows another embodiment of the invention. FIG. 3 shows a layer of noble metal nanoparticles 21 on a three-dimensional surface 20(a) of a three-dimensional substrate structure 20. The layer of noble metal nanoparticles 21 may form an optical coating. In order to increase the amount and/or concentration of noble metal nanoparticles within a specific volume, the three-dimensional substrate structure 20 may possess undulating folds onto which noble metal nanoparticles 21 are adsorbed. The folds may be of all the same scale, or they may be of different scale. For example, in some embodiments, smaller folds might exist on the side of larger folds. The folds may be regularly or irregularly shaped.

In the Example shown in FIG. 3, the undulating surface 20(a) that is coated with noble metal nanoparticles 21 is irradiated with incident light 22 having a first optical signal. The interaction between the incident light 22 and the noble metal nanoparticles 21 is monitored either as reflected light 23 or transmitted light 25. Part of the wavelength range of the incident light 22 is lost through absorption or scattering, and this can be monitored as an absorption spectrum in the reflection mode 24 or the absorption spectrum in the transmission mode 25.

Interactions between capture agents attached to noble metal nanoparticles and target molecules in a fluid can be schematically illustrated in FIGS. 4(a)-4(c). Noble metal nanoparticles 21 are coated with biomolecules such as antibodies 27. When target biomolecules 28 are captured by the antibodies, the absorption spectrum changes (as shown by absorption curves 30, 31 in FIG. 4(c)) whereby both the peak height and wavelength of maximum absorption are both affected.

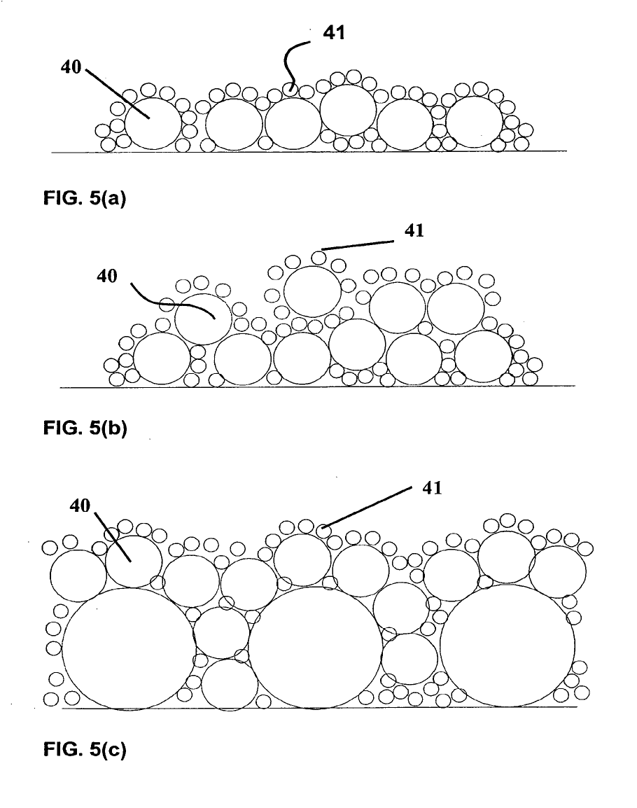

FIGS. 5(a)-5(c) show other three-dimensional substrate structures that can be used in other embodiments of the invention. FIG. 5(a) shows a three-dimensional substrate structure comprising non-noble metal particles 40 including three-dimensional surfaces 40(a). As shown, the noble metal nanoparticles may be over, under, or to the sides of the non-noble metal nanoparticles 40. As shown by FIGS. 5(a)-5(c), the non-noble metal nanoparticles 40 may be of the same or different sizes, and they may be present in one or more layers. For example, as shown in FIG. 5(c), the non-noble metal particles may comprise particles of a first size 40′ and particles of a second sized 40″. In each case, the concentration of noble metal nanoparticles 41 is increased in a specific volume, relative to nanoparticles being on a two-dimensional surface.

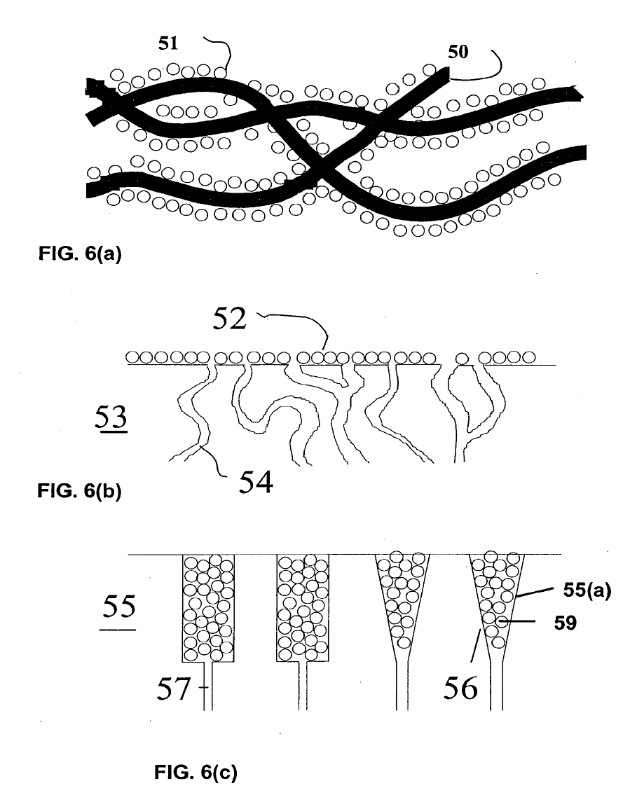

FIGS. 6(a)-6(c) show other embodiments of the invention.

FIG. 6(a) shows a three-dimensional substrate structure 50 in the form of a fabric comprising fibers 50. The fabric may comprise nanowires, a nanomesh, nanotubes, etc. The fabric could be a membrane in some embodiments. The three-dimensional substrate structure 50 may have a three-dimensional surface 50(a) upon which noble metal nanoparticles 51 are present. Pores in the three-dimensional substrate structure 50 allow a fluid including target molecules to flow across the layer of nanoparticles 51. A fluid sample (not shown) comprising target molecules can be forced through the pores of the three-dimensional substrate structure 50 in order to enhance interactions between the capture agents in the fluid on the noble metal nanoparticles 51 and freely suspended target molecules in the fluid.

If the three-dimensional substrate structure 50 comprises a fabric, the fabric may be a woven fabric or a non-woven fabric. It may comprise any suitable material including polymer based materials.

FIG. 6(b) shows another three-dimensional substrate structure 53. The three-dimensional substrate structure 53 includes many nanochannels 54 (and could have nanotubes or nanowires), which give the three-dimensional substrate structure 53 a three-dimensional surface. Noble metal nanoparticles 52 may be adsorbed on top of the substrate structure 53 and may be inside of the nanochannels 54. The nanochannels 54 may be sized so that noble metal nanoparticles 52 are adsorbed on the inside walls of the nanochannels 54. The nanochannels 54 may be regularly or irregularly shaped. In other embodiments, the three-dimensional substrate structure 53 may comprise nanopores.

FIG. 6(c) shows a substrate structure 55 including a number of funnels 57 and cylinders 58 formed therein to provide the three-dimensional substrate structure 55 with a three-dimensional surface 55(a). As shown, the funnels 57 contain layers of noble metal nanoparticles 59. The funnels 57 may be nanofunnels.

The particle based three-dimensional substrate structures shown in FIGS. 5(a)-5(c) and the fabric based three-dimensional substrate structure shown in FIG. 6(a) can be commercially available substrate structures. For example, polystyrene spheres and polyethylene fabrics are commercially available and can be used. The substrate structures shown in FIGS. 6(b) and 6(c) can be formed using techniques know in the art including chemical or physical etching, laser milling, molding, etc.

The use of a three-dimensional substrate structure including a three-dimensional surface provides a number of advantages. The three-dimensional surface provides for an increased amount and/or concentration of noble metal nanoparticles in a detection region of an analytical device as compared to noble metal nanoparticles that are in one layer on a two-dimensional surface of a substrate. This translates into significantly reduced measurement time and a pronounced change in the optical signal.

Analytical Apparatuses Using Mixing

Other embodiments of the invention are directed to methods and analytical apparatuses that effectively mix a small volume of fluid sample (e.g., 50 microliters or less) in a detection region of an analytical device to increase the speed of interaction between target molecules in a fluid and capture agents on noble metal nanoparticles. Increasing the speed of interaction increases the speed of detection.

The inventive mixing mechanisms described herein have a number of advantages. To ensure that proper kinetic data are to be obtained, it is desirable to mix the sample solution sufficiently vigorously so that any potential binding events do not become diffusion-limited. This is typically accomplished by one of the following two methods. In one case, a sample channel is formed over the detection region and the sample is constantly supplied into the detection region. While the flow rate can be controlled very precisely, one problem of this approach is that the longer the reaction time, the more sample is required. Thus, even if the actual sensor surface area may be small, a proportionately large amount of sample is required to continue the flow. It is also possible to re-circulate the sample, but the dead volume of a re-circulating mechanism is not insignificant. The dead volume cannot be reduced readily because simple reduction in size would create a new problem with various seals. The second method for mixing is based on a mechanical stirrer. While this also presents an effective means for mixing, there are mainly two problems associated with it. First, a mechanical mixer cannot be located in the area between the tip of the fiber and the analytical device, because it may block the optical signal so that an extra volume of sample is needed to house the mixing tip. Secondly, mechanical mixing can potentially introduce bubbles, which can interfere with the optical signal. In small structures like micro-wells, bubbles can easily adhere to surfaces, thereby making their removal difficult and thereby interfering with the signal.

In some embodiments, pulses of air (or other fluid) can be introduced into a liquid sample containing target molecules. The pulses of air can be introduced in a repetitive manner. In one exemplary embodiment, a thin tube is immersed into a liquid sample. A small amount of the liquid sample is sucked partly into the thin tube due the capillary action. The tube is connected to an air pump that delivers oscillating air pulses to the tube. As a result, the tip of the tube delivers pulses to the liquid sample, causing the liquid sample to agitate in a repetitive manner. In another exemplary embodiment, the tube may be completely filled with a buffer. The tube can form part of an agitating device and can be partially immersed in the sample as a component that is separate and distinct from the above-described fiber bundle, or can be integrated with the fiber bundle.

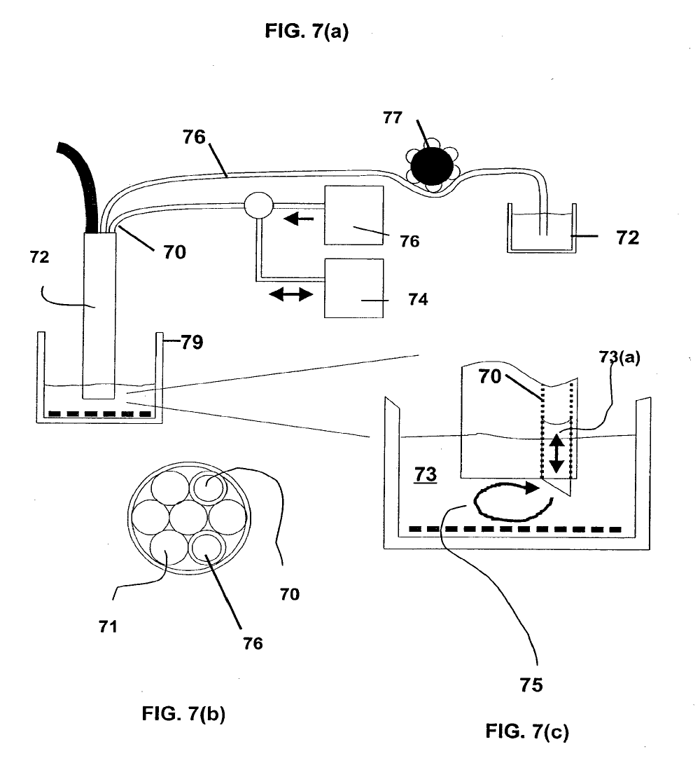

FIGS. 7(a)-7(c) show an embodiment of the invention including an agitating device that includes a pump and a tube that delivers pulses to the sample to agitate it. Part of the tube is present with the fiber bundle comprising parts of the optical fibers that are used to deliver light to and receive light from an analytical device. The setup shown in FIGS. 7(a)-7(c) can include the optical components in described with reference to FIG. 1.

In FIGS. 7(a)-7(c), a hollow tube 70 is bundled together with optical fibers 71 within a single fiber bundle 72. When the fiber bundle 72 is immersed in the sample fluid 73, a small amount of the sample fluid 73(a) rises up the hollow tube 70. A pump 74 connected to the hollow tube 70 sends alternating pulses of air, then the fluid 73(a) in the hollow tube 70 begins to move in a pulsating fashion. Just outside the tube 70, this motion translates into a circular flow 75. When the pump 76 is turned on, air expels the fluid 73(a) out of the hollow tube 70.

Another hollow tube 76 within the fiber bundle 72 may be used to inject a liquid sample into the container 79. The hollow tube 76 may draw from a sample source 72 and may be provided to the container 79 using another pump 77.

The tube diameter, pump volume, oscillation frequency can all be adjusted to maximize the rate of mixing. A microcontroller (not shown) may be connected to the various pumps to provide for a predetermined mixing rate, sample introduction rate, etc.

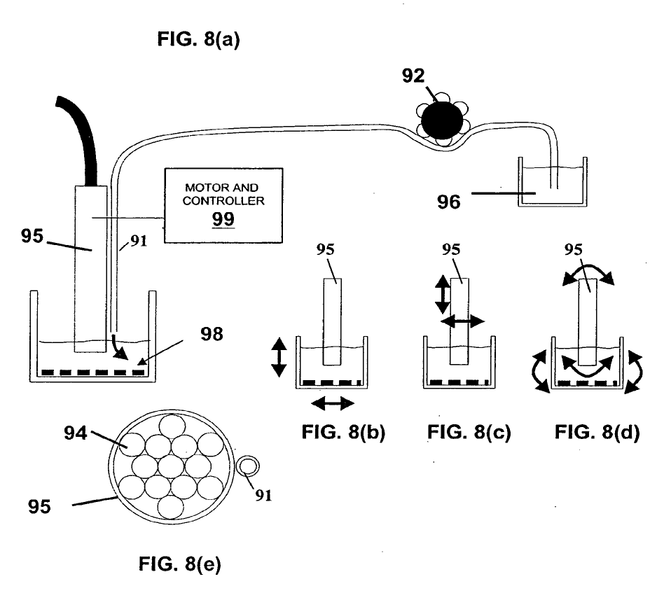

FIGS. 8(a)-8(d) show another embodiment of the invention wherein the agitating device includes a motor and controller 99 coupled to the fiber bundle 95. The illustrated apparatus also includes a sample container 96 that supplied liquid sample to an analytical device 98 in a container using a hollow tube 91 and a pump 92. The fiber bundle 95 comprises optical fibers 94 as in the previously described embodiments. The agitating device in this example may also use any parts (e.g., brackets, linear actuators, etc.) that intervene between the motor and the fiber bundle.

The optical fiber bundle 95 can be used as a mixer by either moving it sideways (as shown in FIG. 8(b)), in the direction parallel to the orientation of the analytical device 98, and/or in a direction perpendicular to the orientation of the analytical device 98 (as shown in FIGS. 8(b)-8(c)). The optical fiber bundle 95 may also be tilted in an oscillating fashion as shown in FIG. 8(d). This mode of mixing is effective with a frequency well below 1 per second with amplitude less than 1 mm if the volume occupied by the immersed portion of the optical fiber is more than 10% of the liquid sample volume.

The optical properties of the analytical device 98 can be monitored constantly during mixing, picking up signals from all of the areas of the analytical device 98 that are illuminated with light from the optical fibers 94. Alternatively, the analytical device may be monitored only at a predetermined area if even slight inconsistencies in the received optical signals are a problem. If the optical fiber bundle 95 is moved vertically in the direction perpendicular to the orientation of the analytical device 98, then optical signals are collected only when the tip of the optical fiber 94 in the fiber bundle 95 is at a predetermined distance away from the analytical device 98.

In some embodiments, mixing a small volume liquid sample can be also achieved by moving the analytical device instead of the optical fiber bundle. The analytical device can be moved sideways or vertically. The analytical device can be also tilted. This mode of mixing is effective with a frequency well below 1 per second with amplitude less than 1 mm if the volume occupied by the immersed portion of the optical fiber is more than 10% of the sample volume. The optical properties of the light obtained from the analytical device might be monitored constantly during mixing, picking up signals from all the area of the sensor substrate that becomes illuminated. Alternatively, the analytical device may be monitored only at a predetermined area if even slight inconsistencies in the monitored optical properties are a problem. If the analytical device moves vertically, then signals are to be collected only when the tip of the optical fiber is at a predetermined distance away from the sensor.

It is also possible to move both the optical fiber bundle and the analytical device with respect to each other. They can move in the same direction, either synchronously or asynchronously. They can also move in different directions. Moreover, the movement can be regular and periodic, or irregular and chaotic.

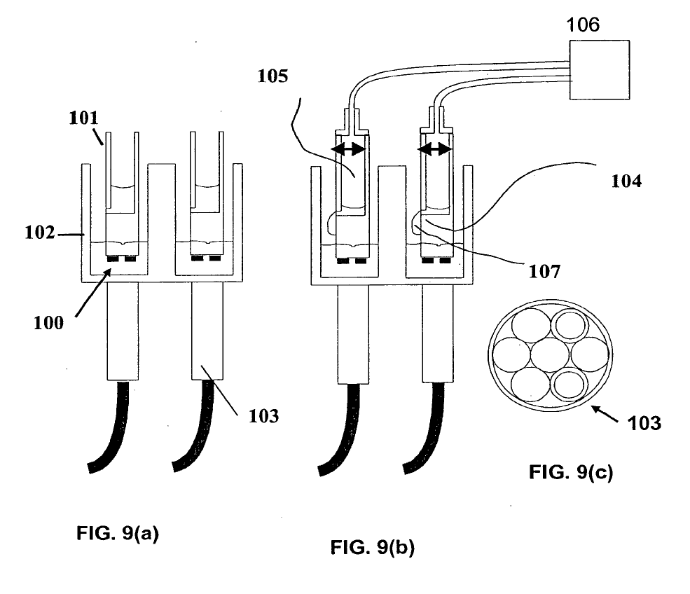

Another alternative analytical apparatus embodiment is shown in FIGS. 9(a)-9(c). FIGS. 9(a)-9(c) show an analytical device comprising nanoparticles 100 that are adsorbed at the tip of a rod 101. The rod 101 can be formed from polymers such as polystyrene, polycarbonate, polymethyl methacrylate (PMMA) or inorganic materials such as glass or silicon. As shown in FIGS. 9(a)-9(b), the tip of the rod 101 can be immersed in a well 102 into which a sample is to be injected. The optical fiber bundle 103 is located at the bottom of the well 102 and is used to provide light to and receive light from the noble metal nanoparticles 100 at the tip of the rod 101 through the transparent well bottom. The tip of the rod 101 can be moved horizontally or vertically to mix the sample in the well 102. The reflection from the tip of the rod 101 may be monitored while the rod 101 is moving or is stationary. A sample 107 can be injected into the well 102 through a gap between the rod 101 and the well wall. Alternatively, a sample 107 can be injected through a hole 104 connecting the hollow interior 105 of the rod 101 with the exterior of the rod 101. If the wall of the connecting hole 104 is hydrophobic and the diameter is sufficiently small, a water-based liquid sample can be held inside the rod 101. When the sample is to be injected into the well 102, a gas, such as air, nitrogen, argon, etc. can be fed into the interior of the rod by a pump 106 to force the sample 107 through the hole 104. This method is particularly effective if there are multiple samples that are to be injected simultaneously into the well 102.

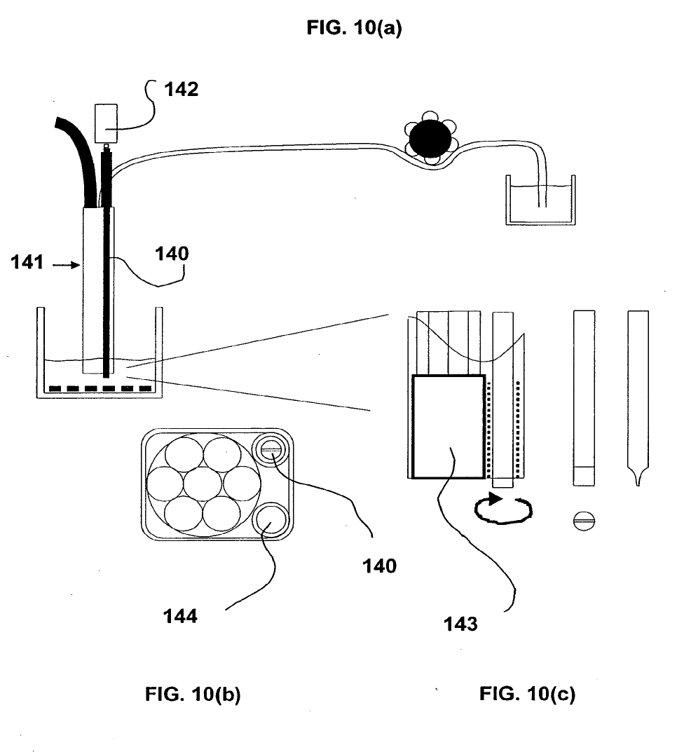

FIGS. 10(a)-10(c) show yet another embodiment of the invention. FIG. 10(a) shows a stirrer 140 that may be inserted into the fluid sample through one or more of the tubes integrated into an optical fiber bundle 141. The stirrer 140 is connected to a motor 142. For efficient mixing, the tip of the stirrer 140 may be shaped like a screw or a flat blade as shown in FIG. 10(c). The stirrer 140 can rotate around its axis or it may move in an oscillatory fashion along the length of the stirrer. The tips of the optical fibers may terminate in a gradient refractive index lens 143 to enhance the light collection efficiency. A sample can be injected through a hollow tube 144 attached to the side of the optical fiber bundle 141.

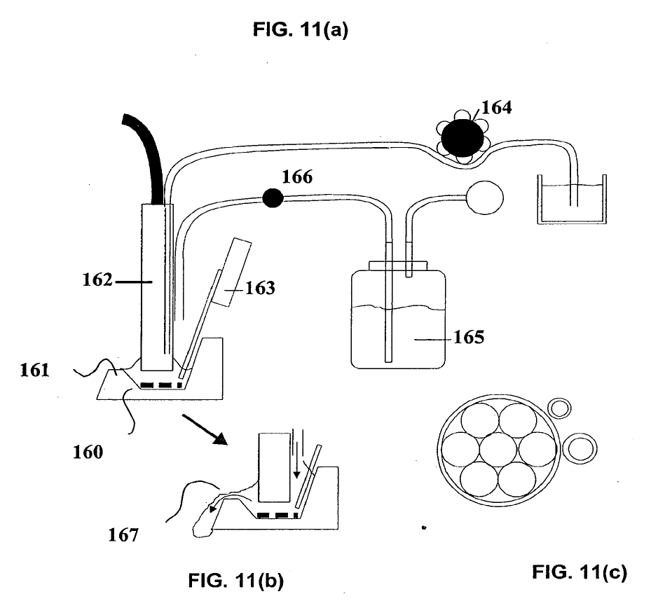

Another analytical apparatus embodiment is shown in FIGS. 11(a)-11(b). Noble metal nanoparticles 160 are adsorbed at the bottom of a shallow well with a low barrier 161. An optical fiber bundle 162 and a mechanical stirrer 163 are inserted into the well. Various fluids are injected through hollow tubes attached to the optical fiber bundle 162. A pump 164 may be employed to directly inject the fluid into the well, or a fluid may be contained in a vessel 165 under pressure. Opening of a valve results in flow of the sample. When the well becomes full as the result of fluid injection, excess fluid overflows over the low barrier 161.

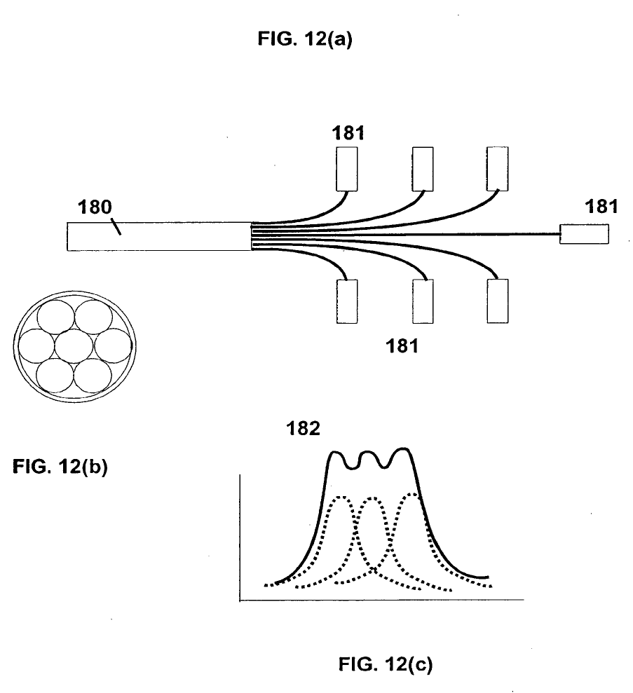

FIGS. 12(a)-12(c) show an optical fiber bundle 180 that can be connected to multiple light emitting diodes 181. These diodes 181 may all emit light of the same wavelength range, or they can be a combination of light emitting diodes giving off light of different wavelength ranges. This arrangement produces an optical spectrum 182 for particular noble metal nanoparticles.

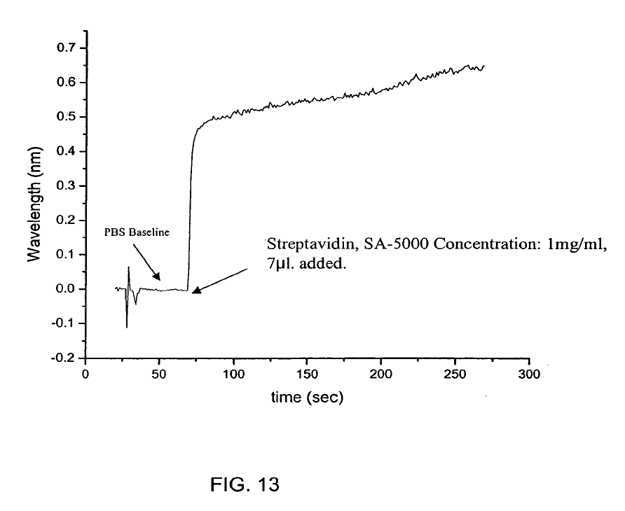

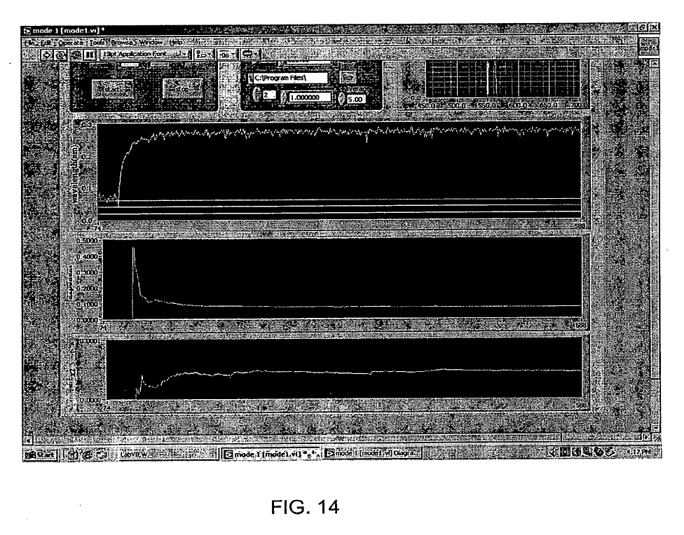

With reference to FIGS. 13-14, a surface adsorbed mono-layer of capped gold particles made up of sub-micron sized polystyrene spheres was formed in accordance with H. Takei, J. Vac. Sci. Technology. B 17, 1906 (1999). The method is based upon the partial aggregation of polystyrene spheres over an Au deposited film that consistently gives samples of excellent uniformity. FIGS. 13-14 show the results of experiments using the techniques in the above article. Both experiments were identical, except for the software that was used. In both cases, the noted wavelength (SPR) change amounts to less than 0.7.

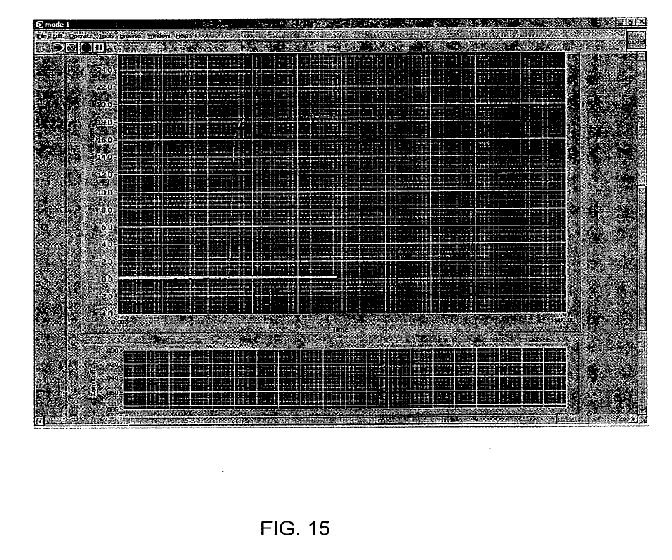

With reference to FIG. 15, an experiment (using the same software as the second example above) was conducted in accordance with an embodiment of the invention. This experiment used a three-dimensional substrate and an agitating device. The wavelength (SPR) change according to the embodiment of the invention greatly exceeded that of examples shown in FIGS. 13 and 14, with an observed shift of 18.8. This resulted in an improvement in sensitivity of over 26-fold.

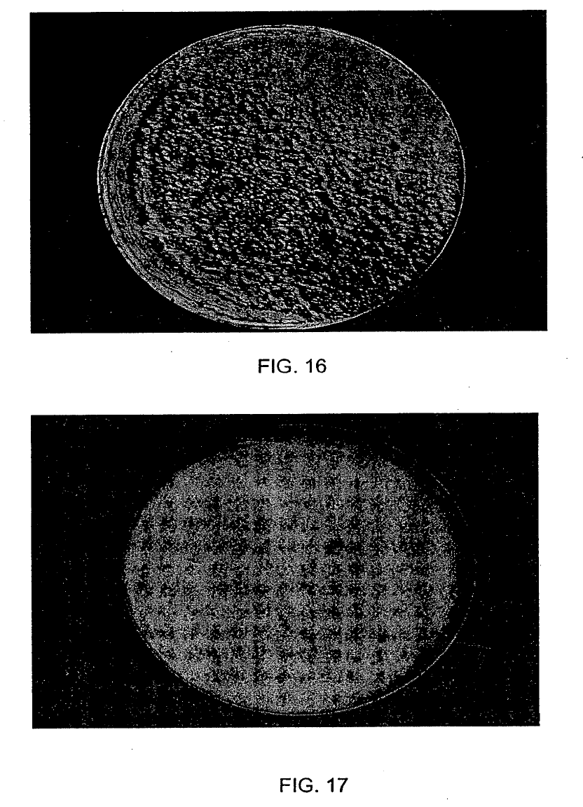

FIG. 16 shows a photograph of an analytical device according to an embodiment of the invention. As shown, the substrate has a bumpy, three-dimensional surface. Nanoparticles (not readily visible) are on the three-dimensional surface. FIG. 17 shows a photograph of an analytical device including a substrate including a flat surface.

Other embodiments of the invention are described in the Examples section below.

### EXAMPLES

The following examples are offered by way of illustration and are not intended to limit the invention in any manner.

### Example I

An analytical apparatus is used and includes an analytical device. The analytical device comprises a light source, an optical fiber bundle equipped with a tip for irradiation and reception, a well possessing surface-adsorbed metal noble nanoparticles, an optical detector used to measure reflectivity as a function of wavelength, a processor for processing optical signals in real time, and a display for showing the result of processed light signals. Light from the light source propagates through the optical fiber bundle. It is emitted from the end of the tip, and the portion of light reflected off from the bottom of the well is picked up by the same tip. It is transmitted to the optical detector whose signal can be processed by the computer. The well contains a fluid that may or may not contain a target molecule. The particles at the bottom of the well are coated with another biomolecule called a capture molecule that possesses a specific affinity toward the target molecule. The binding between the capture molecule and the target molecule triggers an optical event that can be recognized by processing the optical signal from the detector. For irradiation of the analytical apparatus, a pair of optical fiber bundles is used. One optical fiber bundle illuminates the bottom of the well at an angle, and the other optical fiber bundle is used to pick up specularly reflected light. Polarizers may be inserted to selectively make use of polarized light if desired.

### Example II

In another embodiment, the analytical device may comprise a sensor surface that includes an undulating surface onto which gold nanoparticles are adsorbed. The presence of folds in the undulating surface helps to increase the number of gold nanoparticles that can be attached on the surface, thus increasing the sensitivity of the analytical device. Incident light interacts with the layer of gold nanoparticles. Photons within a certain spectrum range interact with the particles, and the portion of the incident light is either absorbed or scattered. This can be monitored as a reflected spectrum or a transmission spectrum. Gold nanoparticles possess capturing molecules with a specific affinity toward a target molecule. When the capturing molecules capture a target molecule, the absorption and/or scattering spectra undergo changes. This can be recognized by monitoring changes in the absorption and/or scattering light as a function of wavelength.

### Example III

### Exemplary Methods for Forming a Three-Dimensional Substrate Structure and Attached Noble Metal Nanoparticles Thereto

In order to create an undulating surface, a dielectric layer of dielectric particles is formed. There are one or more layers of dielectric particles. Polystyrene spheres (1% weight) are placed in a glass test tube, and equal volumes of distilled H₂O and KCl solution (between 0.1 M to 3 M) are added. 100 ml of the mixed solution is placed on a silicon substrate having a surface area of 10 cm². The suspension is allowed to sit for 10 minutes after which a monolayer of adsorbed polystyrene spheres forms on the silicon substrate. Excess spheres are rinsed off by distilled H₂O and the sample is dried in an oven at 60 degrees C. for about 30 minutes.

The function of the KCl solution is to initiate partial aggregation of spheres. Other salts such as NaCl might be substituted for KCl. The dielectric particles do not have to be monodisperse. They may be characterized by more than one size. The surface of these dielectric particles may be modified with an amine or thiol group such that they become attached to gold nanoparticles. When the sphere layer is immersed in a gold nanoparticle suspension, the spheres become automatically coated with gold nanoparticles.

### Example IV

Mixing with a Pulsating Fluid

To provide a mixing mechanism for a small sample volume, an optical fiber bundle equipped with one or more hollow tubes is used. These tubes may be placed together amidst optical fibers or outside the cluster of optical fibers, and these tubes are connected to an air pump that provides and removes air in an alternating sequence, thereby providing pulses of air. When the optical fiber bundle is placed over the analytical device, one only needs enough sample to fill the gap between the ends of the optical fiber bundle and the analytical device. Then a small amount of the sample will be drawn into the hollow tube by capillary action. When the pump is turned on, the sample within the tube begins to oscillate along the length of the tube. At the tube's exit point, the pulsating sample turns into a circular flow. This mechanism provides for the effective mixing necessary for proper kinetic measurements while using minimizing dead volume. Furthermore, the sample within the tube can be easily cleaned out when the pump is set to run in such a way to blow air through the tube. This can be accomplished either by a single pump, which can function in either one of the two modes, or a pair of pumps each dedicated to one of the functions. One advantage of using a pulsating fluid for mixing rather than bubbling air is that the former minimizes interference to the optical signal and reduces the likelihood of drying out the sample (which is in small volume).

### Example V

### Mixing and Measuring a Multiple Number of Samples Simultaneously

In order to measure reactions taking place in the wells of the multiple well plate such as the 96 well plate, one forms gold nanoparticles at the bottom of each well. An optical fiber bundle for monitoring the optical property is inserted into each well. In order to mix samples in every well at the same time, all optical fiber bundles can be moved sideways or vertically, either in a synchronous or random fashion. Monitoring can continue all the time, or alternatively only when the optical fiber bundle is over a particular region of the gold particle coated surface.

In order to measure reactions taking place in the wells of the multiple well plate, it is also possible to prepare the sensor in the form of a pin array. The end of the pin must be either flat or only slightly concave or convex. Gold nanoparticles are coated on the end of each pin. The array is oriented in such a way to point the pins downward and the whole array is lowered into the wells of the multiple well plate. Once placed inside the multiple well plate, the end of each tip is illuminated by an optical fiber bundle through the transparent bottom of the multiple well plate. In order to mix the samples in the wells, the whole array can be moved in an oscillatory fashion either in the horizontal or vertical direction. Alternatively, the plate can be tilted periodically such that as pins at one end of the array are lifted, those on the other end of the array are lowered. The motion can be a combination of any described above. Optical monitoring can proceed throughout the mixing action or only when pins are at some pre-determined location above the optical fiber bundle.

To initiate reactions in a multiple number of wells simultaneously, it is necessary to inject all samples at the same time. One way to facilitate this is described here. When an array of gold nanoparticle coated pins is used, one can select to have a hollow structure within the pin. The bottom of the hollow structure is located at such a position that when the pin is immersed into the well filled with a sample solution, the bottom is still above the sample level. A fine hole connects the interior of the hollow structure to the exterior of the pin; the size of the hole is set small enough and the material of the pin is made hydrophobic enough that when a small amount of liquid is placed within the hollow structure, it will not leak through the hole. Only after pressure is applied, does the liquid flow out through the hole and mix with the sample already inside the well. This method is particularly well suited for injecting different samples into different wells at the same time. Prior to measurement, a fluid-handling robot can be used to fill the hollow structure of every pin with a different sample. Once all the pins are filled, pressure can be applied to the hollow structure of all the pins to drive out all the samples. All the while, only a single air pump is needed rather than many pumps or injectors corresponding in number to the number of all the wells.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed.

Moreover, any one or more features of any embodiment of the invention may be combined with any one or more other features of any other embodiment of the invention, without departing from the scope of the invention.

All patent applications, patents, and publications mentioned above are herein incorporated by reference in their entirety for all purposes. None is admitted to be prior art.

1. An analytical device comprising:
(a) a three-dimensional substrate structure comprising a three-dimensional surface;
(b) a plurality of noble metal nanoparticles on the three-dimensional substrate structure; and
(c) a plurality of capture agents on the noble metal nanoparticles.

2. The analytical device of claim 1 wherein the three-dimensional substrate structure is selected from the group consisting of: a plurality of non-noble metal particles, a substrate comprising an undulating support surface, a fabric or membrane substrate, and a substrate comprising a plurality of wells.

3. The analytical device of claim 1 wherein the noble metal nanoparticles are gold nanoparticles.

4. The analytical device of claim 1 wherein the three-dimensional substrate structure comprises a microscopically rough texture, and wherein each of the noble metal nanoparticles has a size less than about 100 microns.

5. The analytical device of claim 1 wherein the noble metal nanoparticles comprise gold.

6. The analytical device of claim 1 wherein the noble metal nanoparticles comprise silver particles comprising silver oxide layers.

7. The analytical device of claim 1 wherein the three-dimensional substrate structure comprises at least one material selected from the group consisting of: polystyrene, silicon dioxide, titanium dioxide, polymethyl methacrylate (PMMA), and composites thereof.

8. The analytical device of claim 1 wherein the three-dimensional substrate structure comprises a porous material.

9. The analytical device of claim 1 wherein the three-dimensional substrate structure comprises a plurality of layers of fibers.

10. The analytical device of claim 1 wherein the three-dimensional substrate structure comprises a plurality of layers of fibers, wherein the fibers are selected from the group consisting of: polystyrene, nylon, polyethylene, carbon nanotubes, nanowires, nanospheriods, and mixtures thereof.

11.-30. (canceled)
